(19) United States
(12) Patent Application Publication
(10) Pub. No.: US 2023/0000391 A1
(43) Pub. Date: Jan. 5, 2023

(54) METHOD AND SYSTEM FOR ASSESSING A BIOLOGICAL MOVEMENT

(71) Applicant: POLYVALOR, LIMITED PARTNERSHIP, Montréal (CA)

(72) Inventors: Maria Cristina CARMONA DUARTE, Las Palmas de Gran Canaria (ES); Réjean PLAMONDON, Sainte-Julie (CA); Miguel Angel FERRER-BALLESTER, Las Palmas de Gran Canaria (ES)

(21) Appl. No.: 17/778,458

(22) PCT Filed: Nov. 19, 2020
(86) PCT No.: PCT/CA2020/051579

§ 371 Date: May 20, 2022

Related U.S. Application Data
(60) Provisional application No. 62/938,378, filed on Nov. 21, 2019.

(51) Int. Cl. A61B 5/11; G16H 20/30

(52) U.S. Cl. CPC A61B 5/11; G16H 20/30

(57) ABSTRACT

Methods and systems for assessing a biological movement are described. The method comprises obtaining a sequence of samples of a signal from a sensor; determining a set of initial parameters from the signal, the initial parameters comprising trajectory, speed, acceleration and direction data for the sequence of samples; segmenting the signal into first layer segments based on changes in the direction of the trajectory of the sequence of samples; segmenting the first layer segments into second layer segments based on the acceleration data; determining target points of the second layer segments using parameters from the first layer segments and the second layer segments; segmenting the first layer segments into third layer segments based on changes in the trajectory or the acceleration of the sequence of samples; and determining a set of output parameters based on the segmenting and the target points, the output parameters comprising timing information and command information for the biological movement.

### CROSS-REFERENCE TO RELATED APPLICATIONS

The present application claims the benefit of U.S. Provisional Patent Application No. 62/938,378 filed on Nov. 21, 2019, the contents of which are hereby incorporated by reference in their entirety.

### TECHNICAL FIELD

The present disclosure relates generally to the segmentation and analysis of a digital signal that represents a biological movement.

### BACKGROUND OF THE ART

Biological movement can be very complex, and there are many past and ongoing studies in various areas of medicine and human sciences dealing with some of these topics. For example, in neurosciences, handwriting strokes constitute a specific class of rapid human movements and they are used to study neurodegenerative processes involved in diseases such as Parkinson's and Alzheimer's. The early detection of cerebral lesions appears possible by determining slight deviations from the norm, which are not evident by simple visual inspection.

For the purposes of detection, diagnosis, treatment, and research, various tools have been devised for gathering kinematic data from human subjects. Once gathered, a kinematic analysis is performed on the kinematic data to extract valuable information regarding neuromotor skills of the subject. There are many challenging difficulties in obtaining relevant information from the kinematic data. Therefore, improvements are needed.

### SUMMARY

In accordance with a broad aspect, there is provided a computer-implemented method for assessing a biological movement. The method comprises obtaining a sequence of samples of a signal from a sensor; determining a set of initial parameters from the signal, the initial parameters comprising trajectory, speed, acceleration, direction data for the sequence of samples; segmenting the signal into first layer segments based on changes in the direction of the trajectory of the sequence of samples; segmenting the first layer segments into second layer segments based on the acceleration data of the sequence of samples; determining target points of the second layer segments using parameters from the first layer segments and the second layer segments; segmenting the first layer segments into third layer segments based on changes in the trajectory or the acceleration of the sequence of samples; and determining a set of output parameters based on the segmenting and the target points, the output parameters comprising timing information and command information for the biological movement.

In accordance with another broad aspect, there is provided a system for assessing a biological movement. The system comprises a processing unit and a non-transitory computer-readable medium having stored thereon program code. The program code is executable by the processing unit for obtaining a sequence of samples of a signal from a sensor; determining a set of initial parameters from the signal, the initial parameters comprising trajectory, speed, acceleration and direction for the sequence of samples; segmenting the signal into first layer segments based on changes in the direction of the trajectory of the sequence of samples; segmenting the first layer segments into second layer segments based on the acceleration data of the sequence of samples; determining target points of the second layer segments using parameters from the first layer segments and the second layer segments; segmenting the first layer segments into third layer segments based on changes in the trajectory or the acceleration of the sequence of samples; and determining a set of output parameters based on the segmenting and the target points, the output parameters comprising timing information and command information for the biological movement.

In accordance with yet another broad aspect, there is provided a non-transitory computer-readable medium having stored thereon program code for assessing a biological movement. The program code is executable by a processing unit of a computer for obtaining a sequence of samples of a signal from a sensor; determining a set of initial parameters from the signal, the initial parameters comprising trajectory, speed, acceleration and direction for the sequence of samples; segmenting the signal into first layer segments based on changes in the direction of the trajectory of the sequence of samples; segmenting the first layer segments into second layer segments based on the acceleration data of the sequence of samples; determining target points of the second layer segments using parameters from the first layer segments and the second layer segments; segmenting the first layer segments into third layer segments based on changes in the trajectory or the acceleration of the sequence of samples; and determining a set of output parameters based on the segmenting and the target points, the output parameters comprising timing information and command information for the biological movement.

In some embodiments, the signal is reconstructed using the output parameters.

Reconstructing the signal may comprise performing independent signal summations for each of the third layer segments to obtain the velocity and the trajectory of the signal. Reconstructing the signal may comprise applying parameters from the first layer segments, second layer segments, and/or third layer segments to the Kinematic Theory.

In some embodiments, at least some of the target points are outside the trajectory of the sequence of samples.

Features of the systems, devices, and methods described herein may be used in various combinations, in accordance with the embodiments described herein.

### BRIEF DESCRIPTION OF THE DRAWINGS

Reference is now made to the accompanying figures in which:

FIG. 1 is a block diagram of an example system for assessing a biological movement;

FIG. 2 is a flowchart of an example method for assessing a biological movement;

FIGS. 3A-3C schematically illustrate an example of segmenting a sequence of digital samples into first layer segments;

FIGS. 4A-4C schematically illustrate an example of segmenting a sequence of digital samples into second layer segments;

FIGS. 5A-5B schematically illustrate an example of finding target points;

FIGS. 6A-6C schematically illustrate an example of segmenting a sequence of digital samples into third layer segments;

FIGS. 7A-7B schematically illustrate an example of the digital samples as segmented on multiple layers;

FIG. 8 is a graphical illustration representing the distance and angles of a segment; and

FIG. 9 is a block diagram of an example computing device.

It will be noted that throughout the appended drawings, like features are identified by like reference numerals.

### DETAILED DESCRIPTION

There is described herein a method and system for assessing a biological movement. The biological movement is represented by a signal that is digitized and segmented by layer, and parameters are estimated for each layer using a sequence of samples of the digitized signal, as obtained from one or more sensor. Various types of sensors may be used to capture the biological movement, also referred to herein as kinematic data, which may represent handwriting, speech, or natural movement of a subject. Natural movement includes basic locomotion, such as walking, running, climbing or crawling, as well as manipulative movements such as lifting, carrying, throwing and catching.

The method is based on the hypothesis that the central nervous system sends a set of commands to the muscles each time a change in the direction of a movement occurs. Therefore, it is assumed that the information about the trajectories and the movement is loaded in place cells or grid cells of the hippocampus and the medial entorhinal cortex. It is also assumed that each time the direction of a movement changes, the entorhinal cortex transmits a set of commands for this movement sequence to the cerebellum, which is responsible for ensuring that the movement is carried out in a smooth and fluid manner.

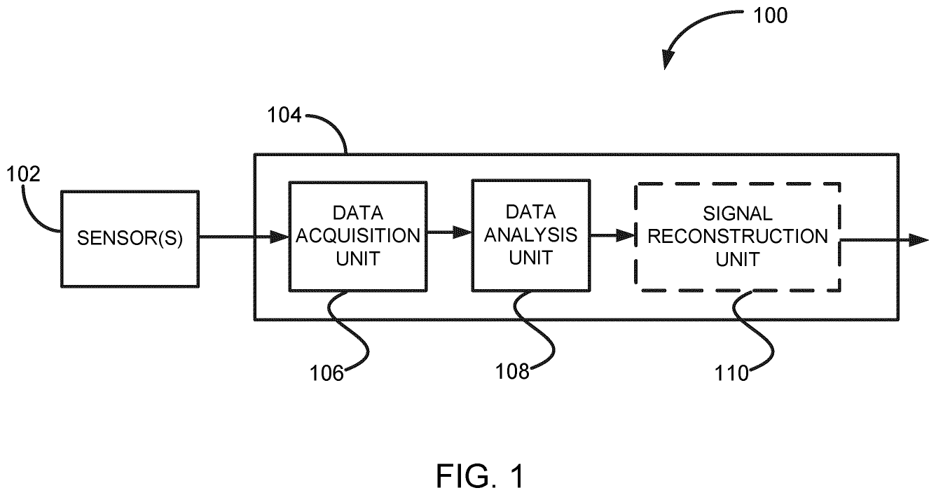

Referring to FIG. 1, there is illustrated an example system 100 for assessing a biological movement of a subject. One or more sensors 102 acquire kinematic data related to handwriting, speech, and/or natural movement of a subject. In some embodiments, the sensor(s) 102 acquire data from 3D motion sensing input devices or cameras that track movement, such as Kinect™, Leap Motion™, and the like.

In some embodiments, the sensor(s) 102 are one or more microphones, to capture speech-related kinematic data. The microphone converts sound into an electrical signal and may be implemented using any known or other microphone technology, such as a dynamic microphone (also called moving-coil microphone), a condenser microphone (also called capacitor microphone or electrostatic microphone), a piezoelectric microphone, a fiber-optic microphone, a laser microphone, a MEMS (microelectrical-mechanical system) microphone, and the like. In some embodiments, the sensor(s) 102 are one or more touchscreen displays of a device, such as a tablet or other computing device, to capture handwriting-related kinematic data. The touchscreen display may be operated by gestures executed by a hand and/or a digital stylus in contact with the touch-screen display or proximate thereto.

In some embodiments, the sensor(s) 102 are one or more accelerometers for capturing natural movement-related kinematic data. For example, the accelerometer may be part of a tablet, a smartwatch, a smartphone, and the like. The accelerometer measures acceleration, i.e. the rate of change of the velocity of an object as it is displaced in space (x, y, z) by the subject. Any technology suitable for measuring proper acceleration, by converting mechanical motion into an electrical signal, may be used. For example, the accelerometer may comprise piezoelectric, piezoresistive and/or capacitive components. In some embodiments, the accelerometer is MEMs-based, such as a thermal accelerometer.

In some embodiments, the sensor(s) 102 comprise an accelerometer and a microphone as a single device, as accelerometers can also be used to record sound. The accelerometer may also incorporate a gyroscope and may be embedded in an Inertial measurement unit (IMU). In some embodiments, sensor(s) 102 are part of a single device used to acquire two or more of the types of kinematic data, for example as described in U.S. Provisional Patent Application No. 62/916,325, the contents of which are hereby incorporated by reference in their entirety. In other embodiments, each type of kinematic data is acquired through a separate device. Various embodiments may apply.

The kinematic data is received at a kinematic assessment device 104, which may comprise a data acquisition unit 106 for acquiring the kinematic data 106 from the sensor(s) 102, and a data analysis unit 108 for analyzing the kinematic data in order to estimate parameters therefrom. Optionally, the kinematic assessment device 104 may also comprise a signal reconstruction unit 110, for reconstructing a digital signal from the parameters estimated by the data analysis unit 108. In some embodiments, the signal reconstruction unit 110 may also be used to reconstruct a synthesized digital signal based on modifications of the estimated parameters. For example, the method described in WO2017216400A1 may be applied using the parameters as estimated by the data analysis unit 108, for generating handwritten text with different degrees of maturity of the subject. In another example, the estimated parameters may be modified to change the age of the subject or to simulate a disease in the subject. Other embodiments may also apply.

The data analysis unit 108 is configured for segmenting the biological movement by layer, so as to provide information about the timing, the displacement, the angles, and other parameters of a lognormal speed profile. Each layer is defined as a grid, where each grid has different distances between the points forming the grid, the first layer having larger distances and the last layer having shorter distance. Segmentation is carried out based on trajectory curvature instead of velocity peaks, as is usually done. Once the parameters of each segment in each layer have been estimated, the trajectory, as well as the velocity and the acceleration profiles can be reconstructed as a summation of overlapping signals. Information about the timing, execution, and storage of information by the central nervous system may thus be obtained.

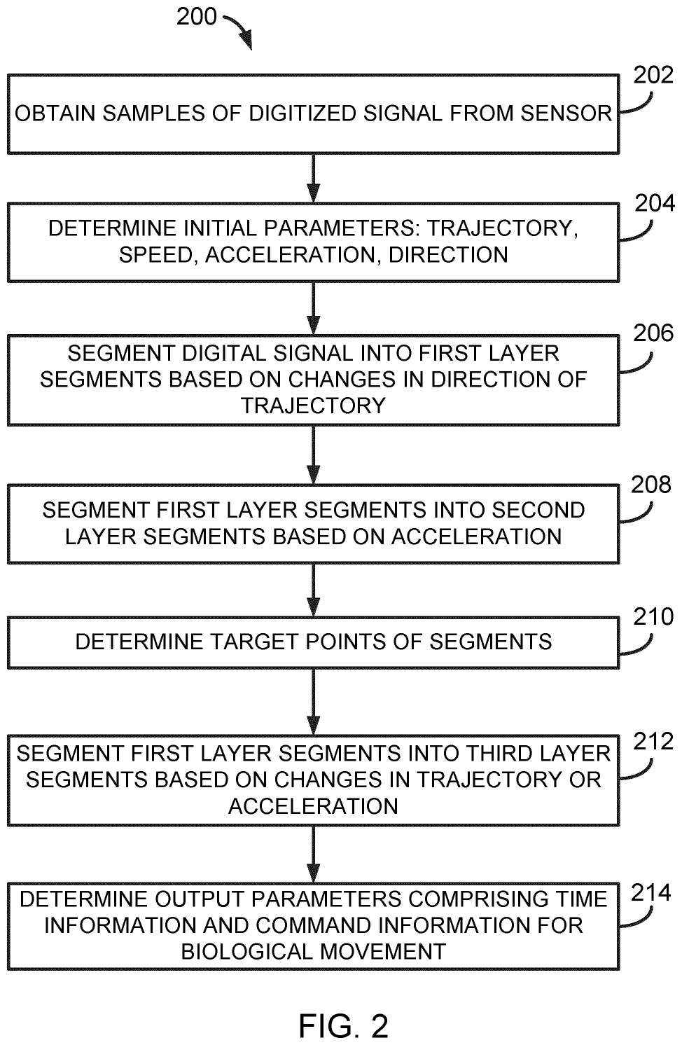

Referring to FIG. 2, there is illustrated an example method 200, as performed by the kinematic assessment device 104. At step 202, a sequence of samples of a signal is received from one or more sensors, such as sensor(s) 102. In some embodiments, the signal received from sensor(s) 102 is an analog signal and the signal is digitized by the data acquisition unit 106. In some embodiments, a digitized signal is received directly at the data acquisition unit 106.

At step 204, a set of initial parameters are determined from the digitized signal. The initial parameters include trajectory, speed, acceleration and direction data for a sequence of samples from the digital signal. Depending on the nature of the kinematic data as received, one or more of the initial parameters can be extrapolated from the kinematic data and the remaining ones of the initial parameters can be calculated therefrom. For example, trajectory data may be received for handwriting-related kinematic data. The trajectory data can be derived to obtain velocity, and the velocity can be derived to obtain acceleration. Similarly, if acceleration data is received, for example for natural movement or speech-related kinematic data, it can be integrated to obtain velocity data, and the velocity data can be integrated to obtain trajectory data.

For the purposes of the present disclosure, the following definitions are provided. The trajectory is defined as:

Pⱼ=(xⱼ,yⱼ,zⱼ,tⱼ) j=1 to N samples.

The velocity is defined as:

$${v}_{j} = \sqrt{{( \frac{Δ {x}_{j}}{Δ {t}_{j}} )}^{2} + {( \frac{Δ {y}_{j}}{Δ {t}_{j}} )}^{2} + {( \frac{Δ {z}_{j}}{Δ {t}_{j}} )}^{2}} ;$$

where the velocity in x, y and z, respectively is:

$${v}_{x j} = \frac{Δ {x}_{j}}{Δ {t}_{j}} , {v}_{yj} = \frac{Δ {y}_{j}}{Δ {t}_{j}} , {v}_{z j} = \frac{Δ {z}_{j}}{Δ {t}_{j}} .$$

The acceleration is defined as:

$${a}_{j} = \frac{Δ {v}_{j}}{Δ t} or {a}_{j} = \sqrt{{( {a}_{x j} )}^{2} + {( {a}_{y j} )}^{2} + {( {a}_{z j} )}^{2}} ;$$

where the acceleration in x, y and z, respectively, is:

$$\begin{matrix}{a}_{x j} = \frac{Δ {v}_{x j}}{Δ t} , & {a}_{y j} = \frac{Δ {v}_{y j}}{Δ t} , & {a}_{z j} = \frac{Δ {v}_{z j}}{Δ t}\end{matrix} .$$

The direction estimated by the sign of the samples is defined as:

Sⱼ=sign(xⱼ₊₁*yⱼ₊₁*zⱼ₊₁−xⱼ*yⱼ*zⱼ).

The direction estimated by the angle ∝ⱼ in degrees where a change of direction is produced between the vectors defined between each sample is given by:

$${∝}_{j} = ( pi - a cos ( \frac{( {Q}_{α j} ) ( {Q}_{b j} )}{‖ {Q}_{α j} ‖ ‖ {Q}_{b j} ‖} ) ) . * 180 / pi ;$$

where Qₐⱼ, and Q_{bj}, are defined as the forward and backward derivative of Pⱼ as:

Qₐⱼ=(xⱼ−x_{j-b1},yⱼ−y_{j-b1},zⱼ−z_{j-b1}) for j=1 to N;

Q_{bj}=(xⱼ−x_{j+b1},yⱼ−y_{j+b1},zⱼ−z_{j+b1}) for j=1 to N;

with ∥Qₐⱼ∥ being the norm of Qₐⱼ, and ∥Q_{bj}∥ the norm of Q_{bj}. The value for b1 may vary depending on the sampling rate and the velocity of the movement. For example, with a sampling rate of 100 Hz and fast movements, b1 may be set to 2.

Once the set of initial parameters are determined, the digitized signal may be segmented into multiple layers. It is assumed that there are at least three layers connected in a same set of grid points. Each layer has a different resolution. Step 206 refers to segmentation in the first layer.

At step 206, the digital signal is segmented into first layer segments using the initial parameters, based on changes in the direction of the trajectory of the sequence of samples. Such changes are denoted as a significant change in Sⱼ and/or ∝ⱼ. The first layer segments are referred to herein as Scripts, which are defined as trajectory data units between two relevant changes of direction points, independently of the number of velocity peaks inside it. The objective of segmenting in the first layer is to find “learned points”, which refers to points that stays stable with age and disease. N₁ points are obtained in the first layer for s Scripts. The output of this segmentation step is the segmentation points d_{1,s} and the sign (or direction) of each script

Segmenting the trajectory comprises finding the relevant changes of direction over the sequence of samples in the first layer, and the points d_{1,s} where there are relevant changes of direction in the trajectory. To this end, the j points (1≤j≤N) where there is a peak on ∝ⱼ are selected. In some embodiments, the peaks that are nearest to a change of sign in Sj, are selected. Other techniques for selecting the relevant samples where a change of direction happens may also be used. In some embodiments, in the case of signatures or digitized signals with different Script sizes and fast velocity changes, second peaks are also found between the two points that define the Script. In some embodiments, the following criteria are applied together to select the peaks: (1) the difference between two selected points is greater than 0.03 s; (2) there is no 0 in velocity in the interval; and (3) the number of angle vectors ∝ⱼ is lower than a threshold or the maximum velocity in the interval is lower than 60% of the velocity of the input signal analyzed. Other criteria may also be used.

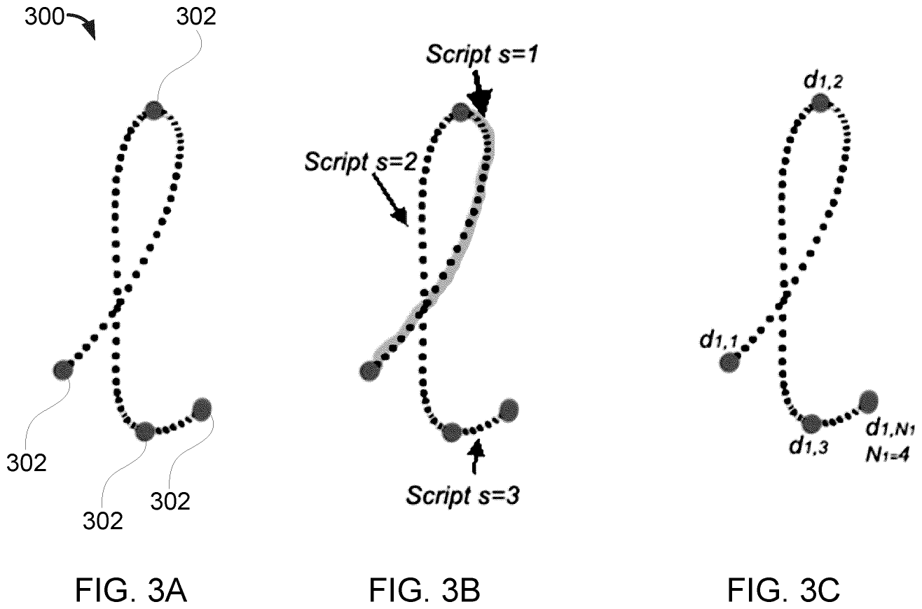

Reference is made to FIGS. 3A-3C, which illustrate an example digitized signal 300 segmented into three Scripts. FIG. 3A illustrates the digitized signal 300 composed of a sequence of samples, with Script points 302 identified therein. FIG. 3B identifies Scripts 1-3. FIG. 3C identifies samples d_{1,1} and d_{1,2} as the start and end points of Script s=1, respectively; samples d_{1,2} and d_{1,3} as the start and end points of Script s=2, respectively; samples d_{1,3} and d_{1,N1} as the start and end points for Script s=3, respectively, where N₁=4 here.

Referring back to FIG. 2, at step 208, the first layer segments are segmented into second layer segments based on acceleration. The second layer segments are referred to herein as Subscripts, and this segmentation step refers to segmentation in a second layer. For each Script, points where a first Script begins to overlap with a second script are identified, and these points are used as endpoints for a Subscript. Segmentation in the second layer results in N₂ new points (d_{2,s,n}), some of which are common with the N₁ points of the first layer. The first index (‘2’) refers to the layer number, the second index (‘s’) refers to the Script number, the third index (‘n’) refers to the Subscript number. The relevant points (d_{2,s,n}) are found in the trajectory by:

$${d}_{2 , s , 2} = {min}_{s ⩽ j ⩽ s + 1} ( {a}_{j} ) ;$$

where d_{2,s,1} is equal to d_{1,s} and d_{2,s,3} equal to d_{1,s+1}. The time increment (Δtd) in this layer can be defined as:

Δtd_{d}_{_{s,s,n}}=t_{d}_{_{s,s,n}}−t_{d}_{_{2,s,n−}}³¹ 2≤n≤N_{2,s}

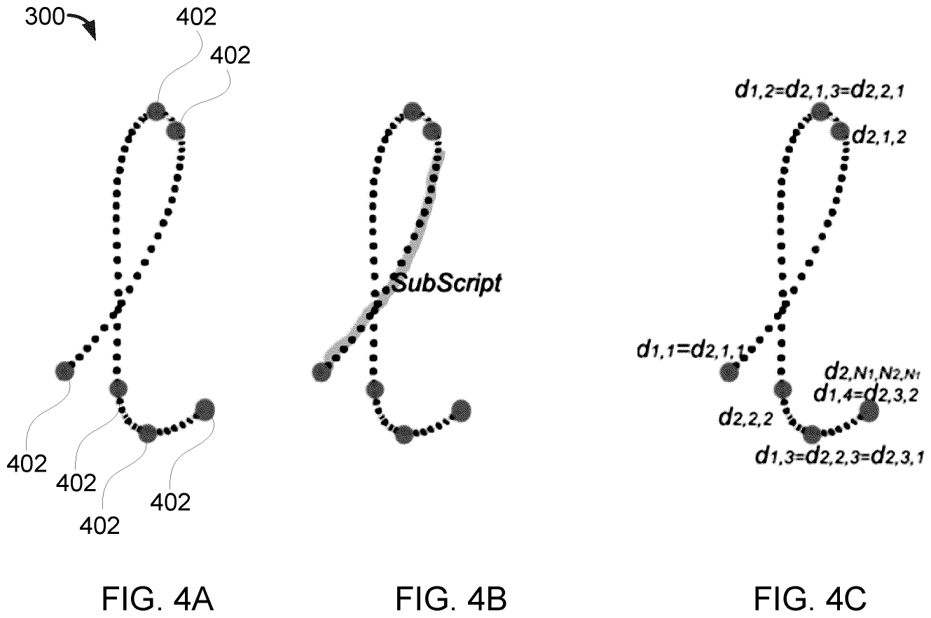

Reference is made to FIGS. 4A-4C, which illustrate an example of the digitized signal 300 segmented into Subscripts. FIG. 4A illustrates the digitized signal 300 with Subscript points 402 identified therein. FIG. 4B illustrates a first of five Subscripts. FIG. 4C identifies samples d_{2,1,1} and d_{2,1,2} as the start and end points of Subscript n=1 respectively; samples d_{2,1,2} and d_{2,2,1} as the end points of Subscript n=2; samples d_{2,1,3} and d_{2,2,2} as the start and end points of Subscript n=3 respectively; samples d_{2,2,2} and d_{2,2,3} as the start and end points of Subscript n=4 respectively; and samples d_{2,3,1} and d_{2,3,2} as the end points of Subscript n=5 respectively.

Due to the overlapping between two Scripts, the “target points” of the trajectory defined by a Script are not in the original trajectory, they are outside of the original trajectory. Target points are not reached due to a change in direction in order to reach a next point. Target points are determined at step 210 of the method 200. First, target points of the Scripts are found using points from the Scripts and Subscripts. Then, target points of the Subscripts are found using the target points from the Scripts.

The target points of the Scripts are referred to as and are found as the intersection between two lines L1 and L2, where L1 is defined by two distinct points (P_{d}_{_{2,s,2}}ₓ, P_{d}_{_{2,s,2}}_{,y}, P_{d}_{_{2,s,2}}_{,z}) and (P_{d}_{_{2,s,2}}_{+1,x}, P_{d}_{_{2,s,2}}_{+1,y}, P_{d}_{_{2,s,2}}_{+1,z}) and L2 is defined by two distinct points

$$( {P}_{{d}_{2 , s , {N}_{2 , s}} + {m}_{1} , x ,} {P}_{{d}_{2 , s , {N}_{2 , s}} + {m}_{1} , y} , {P}_{{d}_{2 , s , {N}_{2 , s}} + {m}_{1} , z} )$$

$$and$$

$$( {P}_{{d}_{2 , s , {N}_{2 , s}} + {m}_{2} , x ,} {P}_{{d}_{2 , s , {N}_{2 , s}} + {m}_{2} , y} , {P}_{{d}_{2 , s , {N}_{2 , s}} + {m}_{2} , z} ) ,$$

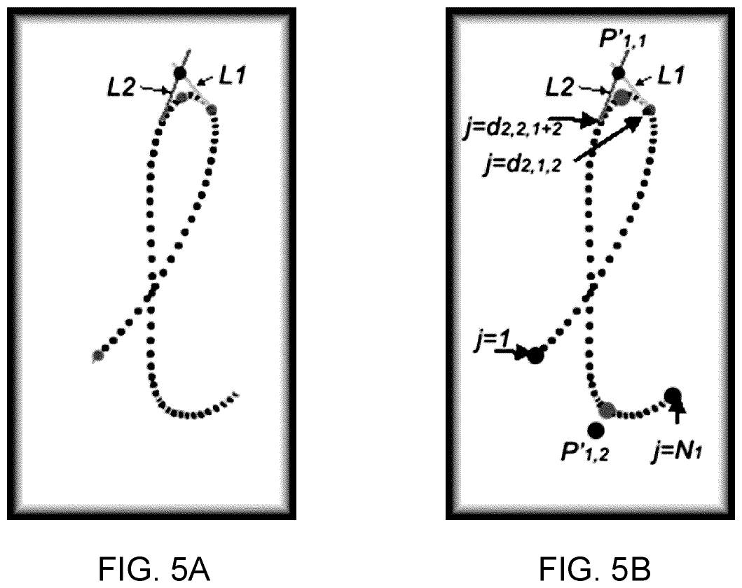

and where m₁ and m₂ are constants that can be used to select suitable target points. The values for m₁ and m₂ may vary depending on the sampling rate. For example, with a sample rate of 100 Hz, m₁ may be set to 2 and m₂ may be set to 3. These values can be optimized depending on the application. Reference is made to FIGS. 5A-5B, where an example is shown of using lines L1 and L2 to find target point P′_{1,1}. The end point d_{1,2} of a first Script may be replaced with target point P′_{1,1}.

Once the target points of the Scripts are found, targets points of the Subscripts are defined as:

$${P}_{2 , s , n}^{′} = \{ \begin{matrix}{P}_{2 , s , 2}^{′} = {P}_{{d}_{2 , s , 2}} & n = 2 \\ {P}_{2 , s , 1}^{′} = {P}_{1 , s}^{′} & n = 1 \\ {P}_{2 , s , {N}_{2 , s}}^{′} = {P}_{1 , s + 1}^{′} & n = 3\end{matrix}$$

Referring back to FIG. 2, at step 212 the first layer segments are segmented into third layer segments based on changes in trajectory or acceleration. The third layer segments are referred to herein as Scriptons, and this segmentation step refers to segmentation in a third layer. N₃ points are found to define I Scriptons in the s Scripts.

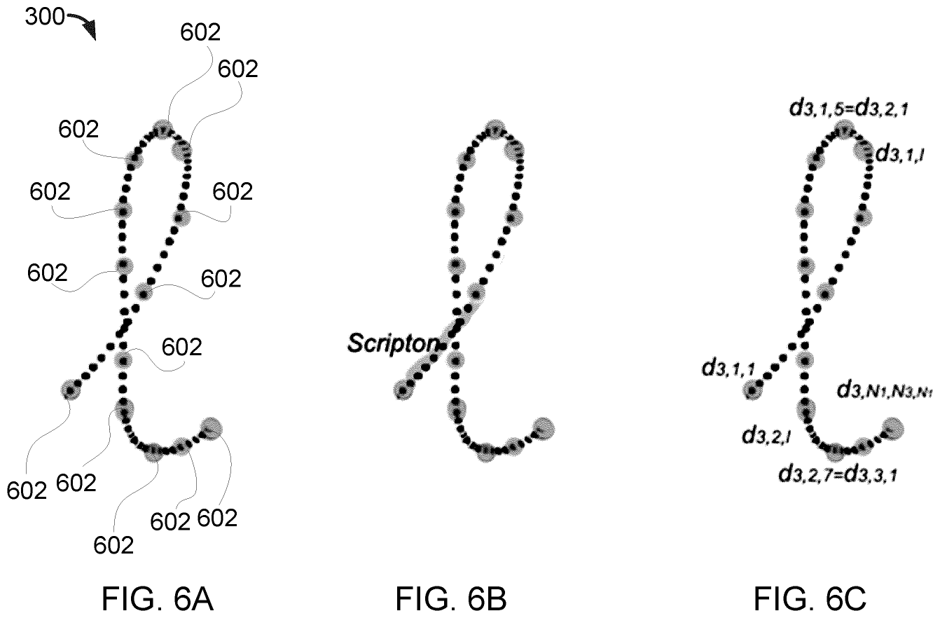

Reference is made to FIGS. 6A-6C, which illustrate the digitized signal 300 segmented into Scriptons. FIG. 6A illustrates the digitized signal 300 with points 602 identified therein. FIG. 6B illustrates a first Scripton as defined by the points 602. FIG. 6C identifies the start and end points of the Scriptons with corresponding labels d_{3,N3,N1}, where N₃=1 to 13 and N₁=1 to 4.

Target points for the Scriptons may also be determined, and are defined as:

$${P}_{3 , s , l}^{′} = \{ \begin{matrix}{P}_{3 , s , l}^{′} = {P}_{{d}_{3 , s , l}} & 2 ⩽ l ⩽ {N}_{3 , s} - 1 \\ {P}_{3 , s , 1}^{′} = {P}_{1 , s}^{′} & l = 1 \\ {P}_{3 , s , {N}_{3 , s}}^{′} = {P}_{1 , s + 1}^{′} & l = 2\end{matrix}$$

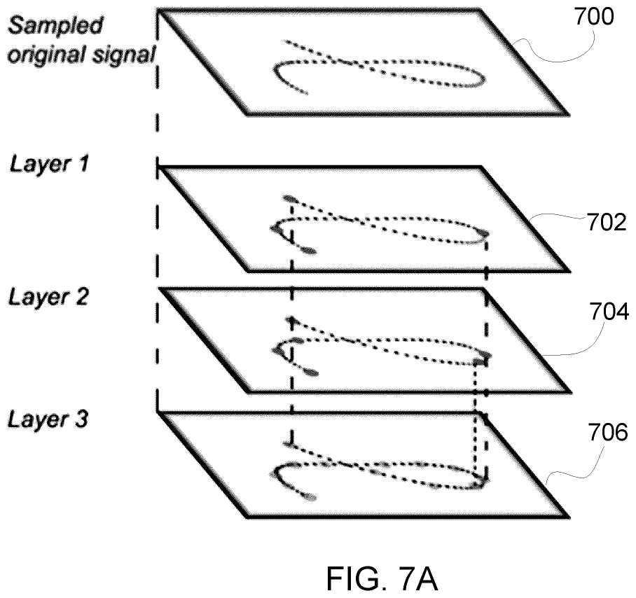

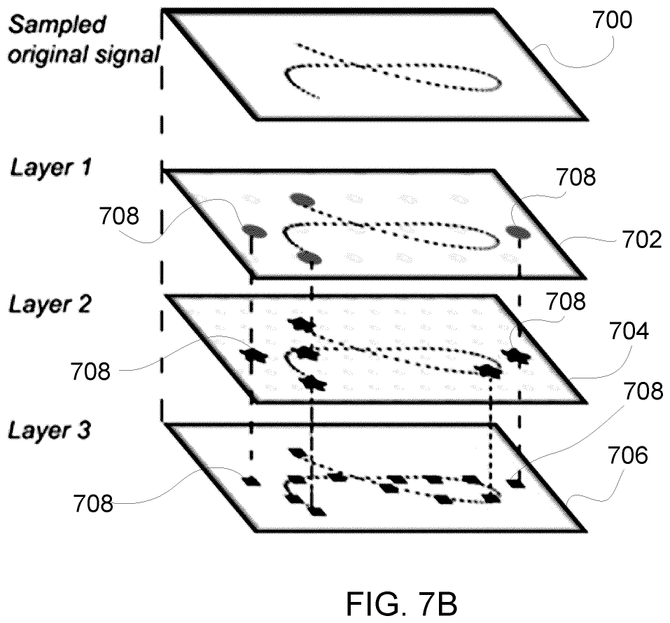

FIG. 7A illustrates the original sampled signal 700 represented in the first layer 702, second layer 704, and third layer 706. Correspondence is also shown between the end points d_{1,s} of the Scripts in the first layer 702, some of the end points d_{2,s,n} of the Subscripts in the second layer 704, and some of the end-points d_{3,s,l} of the Scriptons in the third layer 706. FIG. 7B illustrates target points 708 in the first layer 702, second layer 704, and third layer 706.

Referring back to FIG. 2, at step 214, a set of output parameters comprising timing information and command information for the biological movement are determined based on the previously obtained parameters. Command information refers to an input command from the brain, represented by a distance (D_{3,s,l}) and angles (ø_{3,s,l} and ø′_{3,s,l}). The distance D_{3,s,l} is the intended distance to be covered by a lognormal pulse, as defined in the Kinematic Theory (see Plamondon, R. “A kinematic Theory of Rapid Human Movements. Part I. Movement representation and generation.”, Biol. Cybern. 1995; 72(4): 295-307). Timing information refers to the time (to_{3,s,l}) of occurrence of the command, as instantiated by the central nervous system. The command and timing information may be used to reconstruct analytically the velocity profile and trajectory of the original signal.

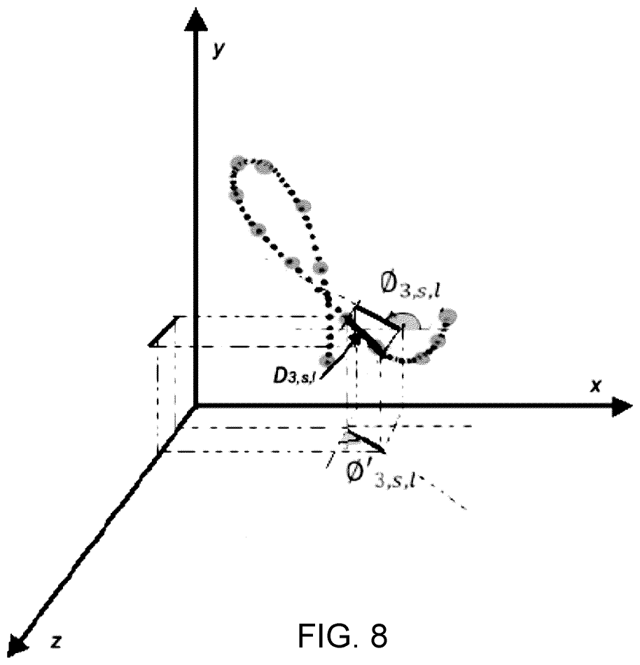

An example is illustrated in FIG. 8 for determining the command information. The distance (which may also be referred to as a displacement given by a velocity vector) may be defined as:

$${D}_{3 , s , l} = \sqrt{{( {P}_{3 , s , l , x}^{′} - {P}_{3 , s , l - 1 , x}^{′} )}^{2} + ( {P}_{3 , s , l , y}^{′} - {P}_{3 , s , l - 1 , y}^{′} ) + {( {P}_{3 , s , l , z}^{′} - {P}_{3 , s , l - 1 , z}^{′} )}^{2}} \\ 2 ⩽ l ⩽ {N}_{3 , s}$$

The angles (ø_{3,s,l} and ø′_{3,s,l}) with respect to the x axis may be defined as:

$${∅}_{{3}_{s , l}} = i \{ \begin{matrix}{tan}^{- 1} \vert ( \frac{{P}_{3 , s , l , y}^{′} - {P}_{3 , s , l - 1 , y}^{′}}{{P}_{3 , s , l , x}^{′} - {P}_{3 , s , l - 1 , x}^{′}} ) \vert First quadrant \\ π - {tan}^{- 1} \vert ( \frac{{P}_{3 , s , l , y}^{′} - {P}_{3 , s , l - 1 , y}^{′}}{{P}_{3 , s , l , x}^{′} - {P}_{3 , s , l - 1 , x}^{′}} ) \vert Second quandrant \\ π + {tan}^{- 1} \vert ( \frac{{P}_{3 , s , l , y}^{′} - {P}_{3 , s , l - 1 , y}^{′}}{{P}_{3 , s , l , x}^{′} - {P}_{3 , s , l - 1 , x}^{′}} ) \vert Third quadrant \\ - {tan}^{- 1} \vert ( \frac{{P}_{3 , s , l , y}^{′} - {P}_{3 , s , l - 1 , y}^{′}}{{P}_{3 , s , l , x}^{′} - {P}_{3 , s , l - 1 , x}^{′}} ) \vert Fourth quadrant\end{matrix}$$

$${{∅}^{′}}_{{3}_{s , l}} = \{ \begin{matrix}π - {tan}^{- 1} \vert ( \frac{\sqrt{{( {P}_{3 , s , l , x}^{′} - {P}_{3 , s , l - 1 , x}^{′} )}^{2} + {( {P}_{3 , s , l , y}^{′} - {P}_{3 , s , l - 1 , y}^{′} )}^{2}}}{{P}_{3 , s , l , z}^{′} - {P}_{3 , s , l - 1 , z}^{′}} ) \vert to ( {P}_{3 , s , l , z}^{′} - {P}_{3 , s , l - 1 , z}^{′} ) > 0 \\ {tan}^{- 1} \vert ( \frac{\sqrt{{( {P}_{3 , s , l , x}^{′} - {P}_{3 , s , l - 1 , x}^{′} )}^{2} + {( {P}_{3 , s , l , y}^{′} - {P}_{3 , s , l - 1 , y}^{′} )}^{2}}}{{P}_{3 , s , l , z}^{′} - {P}_{3 , s , l - 1 , z}^{′}} ) \vert to ( {P}_{3 , s , l , z}^{′} - {P}_{3 , s , l - 1 , z}^{′} ) < 0 \\ \\ \begin{matrix}π / 2 & to ( {P}_{3 , s , l , z}^{′}\end{matrix} - {P}_{3 , s , l - 1 , z}^{′} ) = 0\end{matrix}$$

The sign in each segment is given by:

Sx_{3,s,l}=sign(P′_{d}_{_{l,x}}−P′_{d}_{_{l-1,x}})

Sy_{3,s,1}=sign(P′_{d}_{_{l,y}}−P′_{d}_{_{l-1,y}})

Sz_{3,s,i}=sign(P′_{d}_{_{l,z}}−P′_{d}_{_{l-1,z}})

The time increment for Scriptons (i.e. segments in the third layer) may be defined as:

Δtd_{3,s,l}=t_{d}_{_{3,s,l}}−t_{d}_{_{3,s,l-1}} 2≤l≤N_{3,s}

The initial time where the first lognormal begins in the first layer may be defined as:

$$\begin{matrix}{to}_{1 , s} = {t}_{{d}_{3 , s , l - 1}} + \frac{Δ t {d}_{3 , s , 1}}{2} - 1 \\ {Δ t d}_{1 , s} = {t}_{{d}_{1 , s}} - {t}_{{d}_{1 , s - 1}} 2 ⩽ l ⩽ {N}_{3 , s}\end{matrix}$$

The initial time where each lognormal begins in the third layer may be defined as:

to_{3,s,l}=to_{1,s}+Σₘ₌₁^{l}Δtd_{3,s,m}

In some embodiments, the log response time (σ_{3,s}) is estimated as:

$${σ}_{3 , s , l} = \frac{2. * {max}_{1 < l < {N}_{3} - 1} ( {D}_{3 , s , l} )}{{max}_{{d}_{1 , 1} < j < {d}_{1 , 2}} ( {v}_{j} ) . * \sqrt{2 π}}$$

Optionally, the method 200 comprises a step of reconstructing the digital signal using the output parameters, for example using the signal reconstruction unit 110 of FIG. 1. For this purpose, the velocity of each Script can be calculated as:

$${v}_{{r}_{s , l}} ( t ; {to}_{3 , s , l} ) = {{S}^{′}}_{1 , s} {D}_{3 , s , l} Λ ( t ; {to}_{3 , s , l} , 0 , {{σ}_{3 , s , l}}^{2} ) = {( {S}^{′} )}_{1 , s} \frac{{D}_{3 , s , l}}{{σ}_{3 , s , l} \sqrt{2 π} ( t - {to}_{3 , s , l} )} {e}^{( \frac{- {\vert ln ( t - {to}_{3 , s , l} ) \vert}^{2}}{2 {{σ}_{3 , s , l}}^{2}} )}$$

$$\{ \begin{matrix}{v}_{rx , s , l} ( t ; {to}_{3 , s , l} ) = \vert {v}_{{r}_{s , l}} ( t ; {to}_{3 , s , l} ) \vert cos ( {∅}_{3 , s , l} ) sin ( {∅}_{3 , s , l}^{′} ) \\ {v}_{rx} ( t ) = {v}_{rx} ( t ; {to}_{3 , s , l} ) + {\sum }_{l = 1}^{{N}_{3 , s}} {v}_{rx , s , l} ( t ; {to}_{3 , s , l} )\end{matrix}$$

$$\{ \begin{matrix}{v}_{ry , s} ( t ; {t}_{oj} ) = \vert {v}_{{r}_{s , l}} ( t ; {to}_{3 , s , l} ) \vert sin ( {∅}_{3 , s , l} ) sin ( {∅}_{3 , s , l}^{′} ) \\ {v}_{ry} ( t ) = {v}_{ry} ( t ; {to}_{3 , s , l} ) + {\sum }_{l = 1}^{{N}_{3 , s}} {v}_{y , l} ( t ; {to}_{3 , s , l} )\end{matrix}$$

$$\{ \begin{matrix}{v}_{rz , s} ( t ; {to}_{3 , s , l} ) = \vert {v}_{{r}_{s , l}} ( t ; {to}_{3 , s , l} ) \vert cos ( {∅}_{3 , s , l}^{′} ) \\ {v}_{rz} ( t ) = {v}_{rz} ( t ; {to}_{3 , s , l} ) + {\sum }_{l = 1}^{{N}_{3 , s}} {v}_{z , l} ( t ; {to}_{3 , s , l} )\end{matrix}$$

The analytical trajectory is given by the expressions:

xᵣ(t)=∫vᵣₓ(t)dt+P_{d}_{_{1,1}}_{,x}

yᵣ(t)=∫v_{ry}(t)dt+P_{d}_{_{1,1}}_{,y}

zᵣ(t)=∂v_{rz}(t)dt+P_{d}_{_{1,1}}_{,z}

The analytical acceleration is given by the expressions:

$${a}_{r x} ( t ) = \frac{d {v}_{r x} ( t )}{d t} {a}_{r y} ( t ) = \frac{d {v}_{r y} ( t )}{d t} {a}_{r z} ( t ) = \frac{d {v}_{r z} ( t )}{d t}$$

In some embodiments, the analytical signal as reconstructed is modeled using a single lognormal per Script ‘s’, and lognormal parameters may be estimated for the first layer. The logtime delays μ_{1,s} are defined as:

μ_{1,s}=log(t_{med}_{_{1,s}})

Where t_{med}_{_{1,s}} is the time occurrence of the median of vᵣ_{ₛ}. The distance D_{1,s} of each Script may be defined as:

$${D}_{1 , s} = {\sum }_{l = 1}^{{N}_{3}} {D}_{3 , s , l}$$

The response times are defined as:

$${σ}_{1 , s} = \frac{{D}_{1 , sk}}{{max}_{{d}_{1 , 1} < j < {d}_{1 , 2}} ( {v}_{j} ) * {e}^{{μ}_{{1}_{sk}}} * \sqrt{2 π}}$$

The time occurrence of the lognormal is defined as:

to_{1,s}=tm_{1,s}−e^{(μ}^{_{1,s}}^{-σ}^{_{1,s}}^{²}⁾

Where tm_{1,s} is the time occurrence of the maximum of original velocity in the interval (v) filtered by a low pass filter (for example 16 Hz).

In some embodiments, the analytical signal as reconstructed is modeled using two or more lognormal per Script, and lognormal parameters may be estimated for the second layer. A singular point may be calculated from the end point (d_{2,s,2}), where

$${d}_{1 , s} = {d}_{2 , s , 2} - \frac{{d}_{2 , s , 2}}{2}$$

The logtime delays μ_{2,s,n} are defined as:

μ_{2,s,n}=0 to 1≤n≤N_{2,s}

The distance D_{2,s,n} of each Subscript is defined as:

$${D}_{2 , s , 1} = {\sum }_{l = 1}^{N} {D}_{3 , s , l}$$

$${D}_{2 , s , 2} = {\sum }_{l = N}^{{N}_{3 , s}} {D}_{3 , s , l}$$

The response times are defined as:

$${σ}_{2 , s , 1} = \frac{{D}_{2 , s , 1}}{{max}_{{d}_{2 , s , 1 < j < {d}_{2 , s , 1} + N}} ( {v}_{j} ) . * \sqrt{2 π}}$$

$${σ}_{2 , s , 2} = \frac{{D}_{2 , s , 1}}{{max}_{{d}_{2 , s , 1} + N < j < {d}_{2 , s , {N}_{2 , s}}} ( {v}_{j} ) . * \sqrt{2 π}}$$

The time occurrence of the lognormal is defined as:

to_{2,s,1}=tm_{2,s,1}−1

to_{2,s,1}=tm_{2,s,2}−1

Where tm_{2,s,1} and tm_{2,s,2} are the time occurrences of the maximum of the original velocity (v) in the interval d_{2,s,1}<j<d_{2,s,1}+N or d_{2,s,1}<j<d_{2,s,1}+N respectively, filtered by a low pass filter (for example 16 Hz).

In some embodiments, the method 200 can generate as output timing parameters for each layer, which provides information about the synchronization in the central nervous system:
Δtd_{1,s} is the time increment of each Script in the first layer. Note that it is different to the initial time of the zero crossing in the velocity profile.
Δtd_{2,s,n} is the time increment of each Subscript in the second layer.
Δtd_{3,s,l} is the time increment of each Scripton in the third layer.

The method 200 can also generate target points P′_{1,s}, which may be useful for biometric applications. Also useful for biometric applications are the angles between the segmented trajectories ø_{3,s,1}, ø_{3,s,1} and the displacement or distance in each segment D_{3,s,1}, the latter also providing information regarding neurodegenerative diseases. Statistical analysis of the first layer can lead to σ_{1,s}, to_{1,s}, μ_{1,s}. Statistical analysis of the second layer can lead to σ_{2,s}, to_{2,s}, μ_{2,s}. Statistical analysis of the third layer can lead to σ_{3,s}.

It will be understood that the method 200 allows to separate the biological movement into different layers, each one with a specific timing, independently of the number of peaks that appear in the velocity profile. It also allows recovery of information that cannot be seen in the velocity profile using other approaches. The method 200 may be used (although not exclusively) for any application where the Kinematic Theory has been applied. The method 200 can be used to specify, analyze and monitor the status of the neuromuscular system of a subject, healthy or not, young, mature, or old. For example, it may be used for biometrics, education, sports, rehabilitation, health monitoring, and the like. Some specific and non-limiting examples of applications are handwriting recognition and analysis, writer identification, signature verification, sports movement assessment, learning assessment, gestures biometrics, speech recognition, speech assessment, and animal health assessment. The method 200, due to its speed, can be implemented in small systems, such as tablets, smartwatches, smart phones, and the like. The method 200 may be performed in real time or near real time, due to the speed with which the output parameters can be determined using the steps of the method 200. This allows a means for obtaining feedback for a patient, student, athlete, animal, etc., in a simple manner, prior to performing more complex and in-depth analyses that might require more processing power.

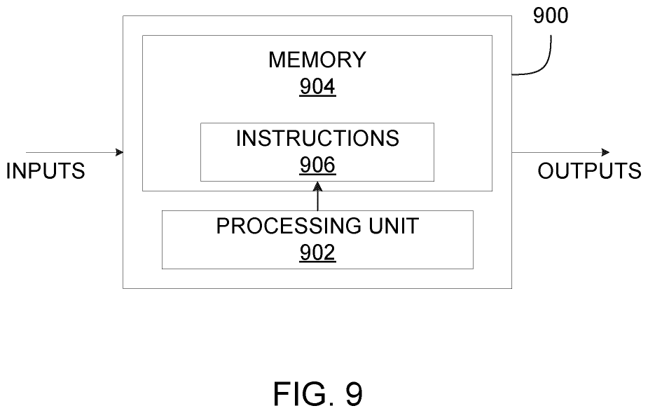

Referring to FIG. 9, an example of a computing device 900 is illustrated for performing some or all of the steps of the method 200. The kinematic assessment device 104, or any other device configured for assessing a biological movement as described herein, may be implemented with one or more computing devices 500. For example, a first computing device 900 may be used to implement the data acquisition unit 106 and a second computing device 900 may be used to implement the data analysis unit 108. Alternatively, a single computing device 900 may be used to implement both the data acquisition unit 106 and the data analysis unit 108. In some embodiments, the signal reconstruction unit 110 is also implemented by the same computing device 900 or a separate computing device 900. Other embodiments may also apply.

The computing device 900 comprises a processing unit 902 and a memory 904 which has stored therein computer-executable instructions 906. The processing unit 902 may comprise any suitable devices configured to implement a method, such that instructions 906, when executed by the computing device 900 or other programmable apparatus, may cause functions/acts/steps as described herein to be executed. The processing unit 902 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory 904 may comprise any suitable known or other machine-readable storage medium. The memory 904 may comprise non-transitory computer readable storage medium, for example, but not limited to, an electronic, magnetic, optical, electromagnetic, infrared, or semiconductor system, apparatus, or device, or any suitable combination of the foregoing. The memory 904 may include a suitable combination of any type of computer memory that is located either internally or externally to the computing device 900, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 904 may comprise any storage means (e.g., devices) suitable for retrievably storing machine-readable instructions 506 executable by processing unit 902.

The methods and systems for assessing a biological movement described herein may be implemented in a high level procedural or object oriented programming or scripting language, or a combination thereof, to communicate with or assist in the operation of a computer system, for example the computing device 900. Alternatively, the methods and systems for assessing a biological movement may be implemented in assembly or machine language. The language may be a compiled or interpreted language. Program code for implementing the methods and systems for assessing a biological movement may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems for assessing a biological movement may also be considered to be implemented by way of a non-transitory computer-readable storage medium having a computer program stored thereon. The computer program may comprise computer-readable instructions which cause a computer, such as the processing unit 902 of the computing device 900, to operate in a specific and predefined manner to perform the functions described herein.

Computer-executable instructions may be in many forms, including program modules, executed by one or more computers or other devices. Generally, program modules include routines, programs, objects, components, data structures, etc., that perform particular tasks or implement particular abstract data types. Typically, the functionality of the program modules may be combined or distributed as desired in various embodiments.

The above description is meant to be exemplary only, and one skilled in the art will recognize that changes may be made to the embodiments described without departing from the scope of the invention disclosed. Still other modifications which fall within the scope of the present disclosure will be apparent to those skilled in the art, in light of a review of this disclosure.

Various aspects of the methods and systems for assessing a biological movement may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. The scope of the following claims should not be limited by the embodiments set forth in the examples, but should be given the broadest reasonable interpretation consistent with the description as a whole.

1. A computer-implemented method for assessing a biological movement, the method comprising:
obtaining a sequence of samples of a signal from a sensor;
determining a set of initial parameters from the signal, the initial parameters comprising trajectory, speed, acceleration and direction data for the sequence of samples;
segmenting the signal into first layer segments based on changes in the direction of the trajectory of the sequence of samples;
segmenting the first layer segments into second layer segments based on the acceleration data of the sequence of samples;
determining target points of the second layer segments using parameters from the first layer segments and the second layer segments;
segmenting the first layer segments into third layer segments based on changes in the trajectory or the acceleration of the sequence of samples; and
determining a set of output parameters based on the segmenting and the target points, the output parameters comprising timing information and command information for the biological movement.

2. The method of claim 1, further comprising reconstructing the signal using the output parameters.

3. The method of claim 2, wherein reconstructing the signal comprises performing independent signal time superimposition and summations for each of the third layer segments to obtain the velocity and the trajectory of the signal.

4. The method of claim 2, wherein reconstructing the signal comprises applying parameters from the first layer segments to the Kinematic Theory.

5. The method of claim 2, wherein reconstructing the signal comprises applying parameters from the second layer segments to the Kinematic Theory.

6. The method of claim 2, wherein reconstructing the signal comprises applying parameters from the third layer segments to the Kinematic Theory.

7. The method of claim 1, wherein at least some of the target points are outside the trajectory of the sequence of samples.

8. The method of claim 1, wherein the set of output parameters are obtained in real time with the acquisition of the signal from the sensor.

9. A system for assessing a biological movement, the system comprising:
a processing unit; and
a non-transitory computer-readable medium having stored thereon program code executable by the processing unit for:
obtaining a sequence of samples of a signal from a sensor;
determining a set of initial parameters from the signal, the initial parameters comprising trajectory, speed, acceleration and direction data for the sequence of samples;
segmenting the signal into first layer segments based on changes in the direction of the trajectory of the sequence of samples;
segmenting the first layer segments into second layer segments based on the acceleration data of the sequence of samples;
determining target points of the second layer segments using parameters from the first layer segments and the second layer segments;
segmenting the first layer segments into third layer segments based on changes in the trajectory or the acceleration of the sequence of samples; and
determining a set of output parameters based on the segmenting and the target points, the output parameters comprising timing information and command information for the biological movement.

10. The system of claim 9, wherein the program code is further executable for reconstructing the signal using the output parameters.

11. The system of claim 10, wherein reconstructing the signal comprises performing independent signal time superimposition and summations for each of the third layer segments to obtain the velocity and the trajectory of the signal.

12. The system of claim 10, wherein reconstructing the signal comprises applying parameters from the first layer segments to the Kinematic Theory.

13. The system of claim 10, wherein reconstructing the signal comprises applying parameters from the second layer segments to the Kinematic Theory.

14. The system of claim 10, wherein reconstructing the signal comprises applying parameters from the third layer segments to the Kinematic Theory.

15. The system of claim 9, wherein at least some of the target points are outside the trajectory of the sequence of samples.

16. The system of claim 9, wherein the set of output parameters are obtained in real time with the acquisition of the signal from the sensor.
